(19) United States
(12) Patent Application Publication
(10) Pub. No.: US 2019/0380596 A1
(43) Pub. Date: Dec. 19, 2019

(54) BIOLOGICAL INFORMATION MEASURING APPARATUS, METHOD AND PROGRAM

(71) Applicants: OMRON CORPORATION, Kyoto (JP); OMRON HEALTHCARE CO., LTD., Kyoto (JP)

(72) Inventors: Tsuyoshi KITAGAWA, Kyoto (JP); Shingo YAMASHITA, Kyoto (JP)

(21) Appl. No.: 16/554,874

(22) Filed: Aug. 29, 2019

Related U.S. Application Data
(63) Continuation of application No. PCT/JP2018/009569, filed on Mar. 12, 2018.

(30) Foreign Application Priority Data
Mar. 15, 2017 (JP) 2017-050631

(51) Int. Cl. A61B 5/0235; A61B 5/022

(52) U.S. Cl. CPC A61B 5/0235; A61B 5/02116; A61B 5/02225; A61B 5/02241

(57) ABSTRACT

A biological information measuring apparatus includes a sensing device and a calibration device. The calibration device includes a transmitter transmitting, to the sensing device, data including the first biological information, and calibration identification information. The sensing device includes a determination unit determining whether the calibration identification information is information of a calibration device corresponding to the sensing device; a detector detecting a pulse wave; and a calculator, when the calibration identification information is information of a corresponding calibration device, calibrating the pulse wave based on the first biological information, and calculating second biological information based on the calibrated pulse wave.

### CROSS-REFERENCE TO RELATED APPLICATIONS

This application is a Continuation Application of PCT Application No. PCT/JP2018/009569, filed Mar. 12, 2018 and based upon and claiming the benefit of priority from Japanese Patent Application No. 2017-050631, filed Mar. 15, 2017, the entire contents of all of which are incorporated herein by reference.

### FIELD

The present invention relates to a biological information measuring apparatus that continuously measures biological information, a method therefor, and a program therefor.

### BACKGROUND

The development of sensor technology has brought about an environment which allows high-performance sensors to be easily used, and it is getting more and more important in medical treatment to detect biological abnormalities early by utilizing biological information and to put it to use in treatment.

There is known a biological information measuring apparatus capable of measuring biological information such as pulse and blood pressure using information detected by a pressure censor being brought in direct contact with a living body site through which an artery such as a radial artery of the wrist extends (see, for example, Jpn. Pat. Appln. KOKAI Publication No. 2004-113368).

The blood pressure measuring apparatus described in Jpn. Pat. Appln. KOKAI Publication No. 2004-113368 calculates a blood pressure value using a cuff at a site different from a living body site which the pressure sensor is brought in contact with, and generates calibration data from the calculated blood pressure value. By calibrating the pressure pulse wave detected by the pressure sensor using the generated calibration data, the blood pressure value is calculated per pulse.

The blood pressure measuring apparatus described in Jpn. Pat. Appln. KOKAI Publication No. 2004-113368 is too large in size to increase the measurement accuracy. Furthermore, this blood pressure measuring apparatus is Premised on operation performed in a limited environment by a specific person. This makes it difficult to use the apparatus for daily medical care or at home. In addition, this blood pressure measuring apparatus requires a large volume of tubes and wiring which are burdensome, and therefore not practical for use on a daily basis or during sleep.

### SUMMARY

According to a first aspect of the present invention, a biological information measuring apparatus comprises a sensing device and a calibration device. The calibration device comprises: a measuring unit that measures first biological information intermittently; and a transmitter that transmits, to the sensing device, data including the first biological information, and calibration identification information which is identification information of the calibration device. The sensing device comprises: a receiver that receives the data and the calibration identification information; a determination unit that determines whether the calibration identification information is information of a calibration device corresponding to the sensing device; a detector that detects a pulse wave in a temporally continuous manner; and a calculator that, when the calibration identification information is information of the corresponding calibration device, calibrates the pulse wave based on the first biological information, and calculates second biological information based on the calibrated pulse wave.

According to a second aspect of the present invention, the sensing device further comprises a sensor memory device that pre-stores identification information of the corresponding calibration device, and when the calibration identification information is included in the identification information stored in the sensor memory device, the determination unit determines that the calibration identification information is information of the corresponding calibration device.

According to a third aspect of the present invention, the sensing device further comprises a sensor registration unit that registers identification information of the corresponding calibration device, and when the calibration identification information is included in the identification information registered in the sensor registration unit, the determination unit determines that the calibration identification information is information of the corresponding calibration device.

According to a new aspect different from the third aspect of the present invention, the biological information measuring apparatus further comprises a transmitter that, when the calibration identification information determined to be not information of the calibration device corresponding to the sensing device, transmits an indication of invalid apparatus to the calibration device.

According to a fourth aspect of the present invention, the sensing device further comprises a sensor memory that stores identification information of the calibration device corresponding to the sensing device; and a sensor pairing unit that emits first radio wave, receives a second radio wave, and pairs with a calibration device emitting the second radio wave if identification information included in the second radio wave matches information in the sensor memory. The calibration device further comprises: a calibration memory that stores identification information of the sensing device corresponding to the calibration device; and a calibration pairing unit that emits the second radio wave, receives the first radio wave, and pairs with a sensing device emitting the first radio wave if identification information included in the first radio wave matches information stored in the calibration memory.

According to a fifth aspect of the present invention, the sensing device further comprises a sensor cancellation detector that determines whether pairing has been cancelled, and instructs the sensor pairing unit to start pairing when it is determined that pairing has been cancelled, and the calibration device further comprises a calibration cancellation detector that determines whether pairing has been cancelled, and instructs the calibration pairing unit to start pairing when it is determined that pairing has been cancelled.

According to a sixth aspect of the present invention, the measuring unit measures the first biological information with higher accuracy than that of second biological information obtained from the detector.

According to a seventh aspect of the present invention, the detector detects the pulse wave for each pulse, and the first biological information and the second biological information indicate blood pressures.

According to the first aspect of the present invention, the sensing device comprises a detector that detects a pulse wave in a temporally continuous manner, and a calculator that, when calibration identification information is information of the corresponding calibration device, calibrates the pulse wave based on first biological information and calculates second biological information from the calibrated pulse wave. The sensing device is separated from the calibration device. Thus, the sensing device is made compact, allowing the sensor to be placed at a position where the pulse wave can be acquired more reliably. The calibration device measures first biological information intermittently, and transmits, to the sensing device, data including the first biological information and calibration identification information which is identification information of the calibration device. This makes it possible to calculate biological information with high accuracy from the pulse waves so that a user can easily obtain biological information with high accuracy. Furthermore, the measuring unit only measures in an intermittent manner. This reduces the time during which a user is interrupted by the measuring unit. Moreover, since the calibration device is independently provided, the calibration device can be mounted at a position appropriate for calibration with ease, regardless of the placement of the sensing device. In addition, the sensing device acquires calibration identification information which is identification information of the calibration device, and determines whether the calibration identification information is information of the calibration device corresponding to the sensing device. When the calibration identification information is information of the corresponding calibration device, the sensing device confirms the calibration deice to be paired with, and can receive blood pressure data from the calibration device worn by the same person and meeting certain criteria, for example. This ensures that the blood pressure is always measured with the same pair of sensing device and calibration device no matter how many calibrations are made.

According to the second aspect of the present invention, the sensing device further comprises a sensor memory device that pre-stores identification information of the corresponding calibration device, and when the calibration identification information which is identification information of the calibration device included in the identification information stored in the Sensor memory device, the determination unit of the sensing device determines that the calibration identification information is information of the corresponding calibration device. This makes it possible to perform blood pressure measurement with the calibration device that is supposed to be paired with the sensing device. For example, if the sensing device pre-stores identification information of a calibration device with higher accuracy than a certain criterion, blood pressure can be measured with high accuracy.

According to the third aspect of the present invention, the sensing device further comprises a sensor registration unit that registers identification information of the corresponding calibration device, and when calibration identification information which is identification of the calibration device is included in the identification information registered in the sensor registration unit, the determination unit determines that the calibration identification information is information of the corresponding calibration device. This allows reliable data exchange only between the sensing device and the calibration device paired with each other. Therefore, the sensing device and the calibration device no longer measure different living bodies, and it is possible to reliably measure biological information with the sensing device and the calibration device worn by the same biological body.

Furthermore, according to the above-mentioned new aspect different from the third aspect of the present invention, when it is determined that the calibration identification information is not the information of the calibration device corresponding to the sensing device, the transmitter transmits an indication of invalid apparatus to the calibration device. Thus, both the calibration device and the sensing device can recognize that this calibration device cannot be paired with the sensing device that has transmitted the calibration identification information. The calibration device and the sensing device can determine which apparatus cannot be paired with, and this eliminates meaningless data exchange between the calibration device and the sensing device.

According to the fourth aspect of the present invention, the sensing device further comprises: a sensor memory that stores identification information of the calibration device corresponding to the sensing device; and a sensor pairing unit that emits a first radio wave, receives a second radio wave, and pairs with a calibration device emitting the second radio wave if identification information included in the second radio wave matches information in the sensor memory. The calibration device further comprises: a calibration memory that stores identification information of the sensing device corresponding to the calibration device; and a calibration pairing unit that emits the second radio wave, receives the first radio wave, and pairs with a sensing device emitting the first radio wave if identification information included in the first radio wave matches information stored in the calibration memory. Therefore, if a failure occurs in either the sensing device or the calibration device, pairing can be newly established based on the identification information of the partner apparatus stored in the apparatus having no failure. This makes it possible to immediately resume and continue measurement of the biological information after failure. By registering only the apparatus whose accuracy has been ensured in the sensor memory and the calibration memory, it is possible to continue measurement of biological information with high accuracy even if a failure occurs in either the sensing device or the calibration device.

According to the fifth aspect of the present invention, the sensing device further comprises a sensor cancellation detector that determines whether pairing has been cancelled, and instructs the sensor pairing unit to start pairing when it is determined that pairing has been cancelled, and the calibration device further comprises a calibration cancellation detector that determines whether pairing has been cancelled, and instructs the calibration pairing unit to start pairing when it is determined that pairing has been cancelled. Therefore, even when pairing is cancelled due to deterioration in communication status, etc., because both the sensing device and the calibration device detect the pairing cancellation and start pairing, the connection between the sensing device and the calibration device can be resumed with a small number of disconnections. Thus, even when the communication status is deteriorated and pairing is cancelled, the measurement of the biological information can be resumed immediately if the communication status is improved.

According to the sixth aspect of the present invention, the measuring unit measures first biological information with higher accuracy than that of second biological information obtained from the detector, and the biological information with high accuracy is obtained from the measuring unit and is calibrated. This ensures the accuracy of biological information obtained based on a pulse wave from the detector. Therefore, it becomes possible to calculate biological information with high accuracy in a temporally continuous manner.

According to the seventh aspect of the present invention, the detector detects the pulse wave for each pulse, and the first biological information and the second biological information indicate blood pressures. This enables the biological information measuring apparatus to measure blood pressure for each pulse wave per pulse in a temporally continuous manner.

That is, according to each aspect of the present invention, it is possible to provide a biological information measuring apparatus, method, and program that are capable of acquiring accurate information while calibrating biological information in a temporally continuous manner with the apparatus being worn constantly.

### BRIEF DESCRIPTION OF THE DRAWINGS

FIG. 1 is a block diagram showing a blood pressure measuring apparatus according to a first embodiment;

FIG. 2 is a view showing an example in which the blood pressure measuring apparatus of FIG. 1 is worn on a wrist;

FIG. 3 is a view showing another example in which the blood pressure measuring apparatus of FIG. 1 is worn on the wrist;

FIG. 4 is a view shoving a time course of a cuff pressure and a pulse wave signal by an oscillometric technique;

FIG. 5 is a view showing a time variation of a pulse pressure per pulse and a pulse wave of one of pulses;

FIG. 6 is a flowchart showing a calibration method;

FIG. 7 is a flowchart when data is transmitted from a sensing device to a calibration device of the blood pressure measuring apparatus of FIG. 1;

FIG. 8 is a block diagram showing a blood pressure measuring apparatus according to a second embodiment;

FIG. 9 is a flowchart in which identification information is exchanged between a sensing device and a calibration device of the blood pressure measuring apparatus of FIG. 8;

FIG. 10 is a flowchart when data is transmitted from the sensing device to the calibration device of the blood pressure measuring apparatus of FIG. 8;

FIG. 11 is a block diagram showing a blood pressure measuring apparatus according to a third embodiment;

FIG. 12 is a flowchart showing an operation in which the sensing device of the blood pressure measuring apparatus of FIG. 8 identifies the calibration device;

FIG. 13 is a block diagram showing a blood pressure measuring apparatus according to a fourth embodiment;

FIG. 14 is a flowchart showing an operation in which a sensing device and a calibration device of the blood pressure measuring apparatus of FIG. 13 are paired with each other;

FIG. 15 is a block diagram showing a blood pressure measuring apparatus according to a fifth embodiment; and

FIG. 16 is a flowchart showing an operation in which pairing of the blood pressure measuring apparatus of FIG. 15 is cancelled and resumed.

### DETAILED DESCRIPTION

Hereinafter, a biological information measuring apparatus, method, and program according to embodiments of the present invention will be described with reference to the accompanying drawings. In the following embodiments, the portions given the same numbers operate similarly, and the overlapping description will be omitted.

The present embodiments have been made in view of the above circumstances, and aim to provide a biological information measuring apparatus capable of acquiring accurate information while calibrating biological information in a temporally continuous manner with the apparatus being worn constantly, a method therefor, and a program therefor.

### First Embodiment

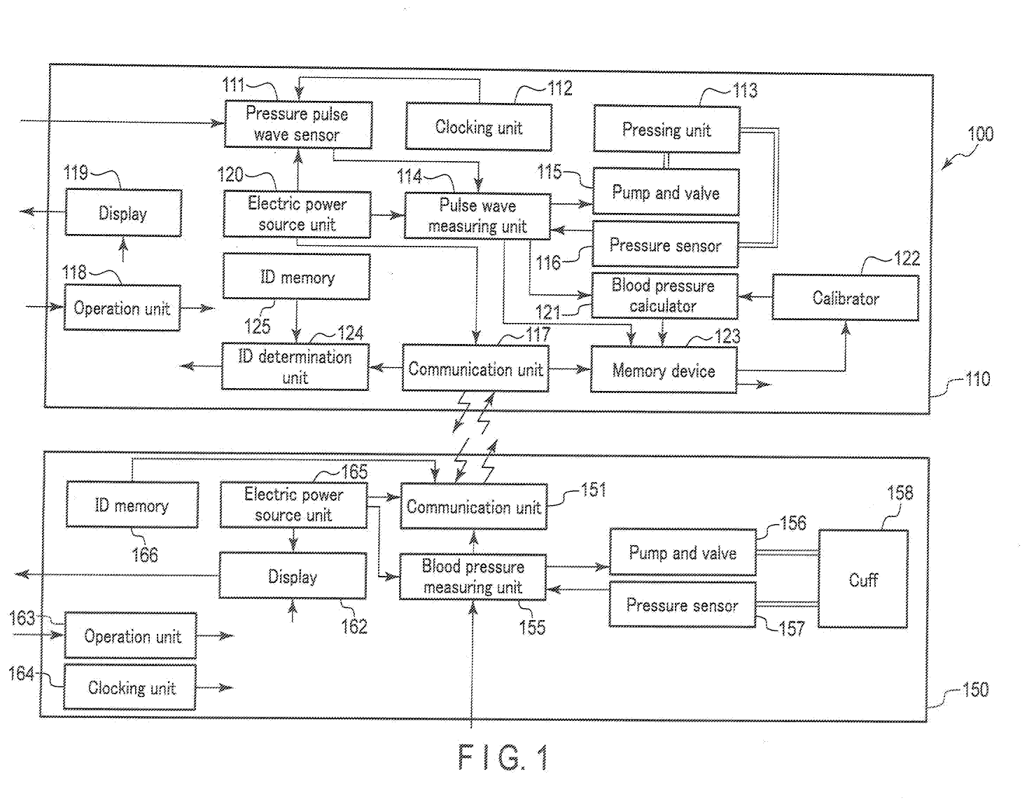

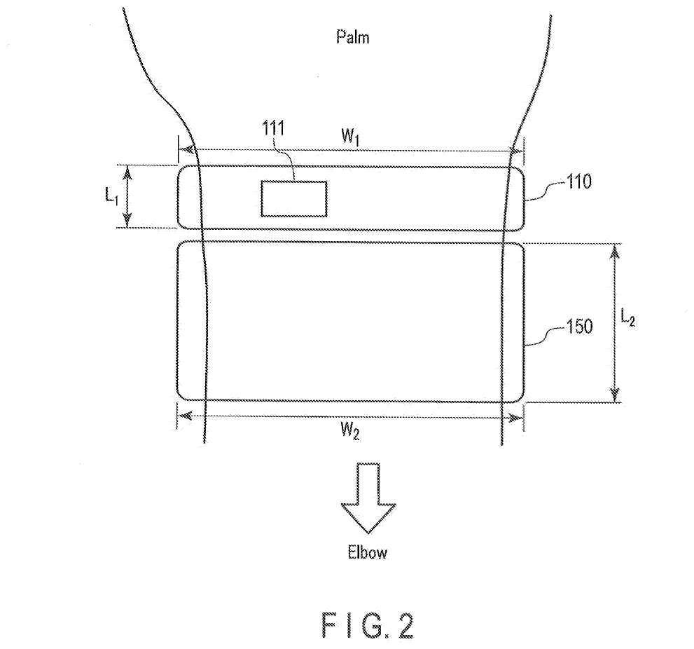

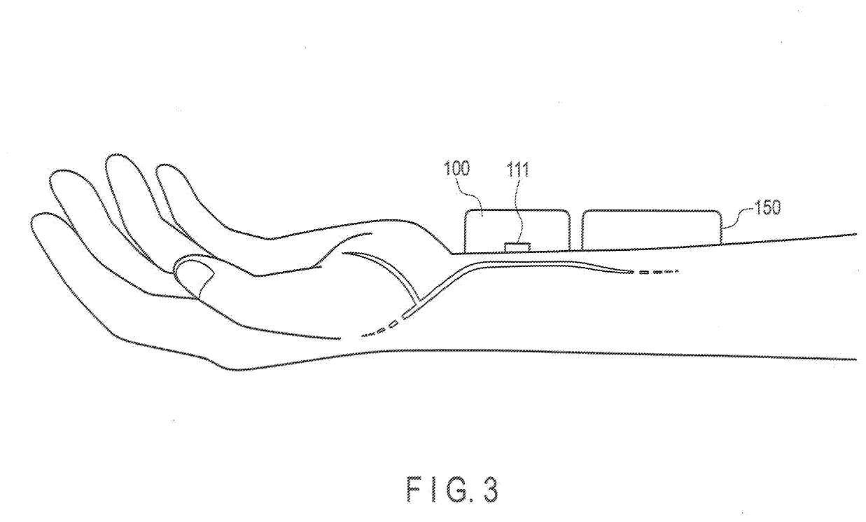

A blood pressure measuring apparatus 100 according to the present embodiment will be described with reference to FIGS. 1, 2, and 3. FIG. 1 is a functional block diagram of the blood pressure measuring apparatus 100, and illustrates details of a sensing device 110 and a calibration device 150. FIG. 2 shows an example in which the blood pressure measuring apparatus 100 is worn on the wrist, and is a schematic perspective view seen from above the palm. A pressure pulse wave sensor 111 is placed on the side of the sensing device 110. FIG. 3 is a schematic perspective view conceptually illustrating the blood pressure measuring apparatus 100 when worn, as seen from the lateral side of the palm (i.e., the direction in which the fingers are aligned when the hand is open). FIG. 3 shows an example in which the pressure pulse wave sensor 111 is placed orthogonal to the radial artery. Although in FIG. 3, the blood pressure measuring apparatus 100 appears to be simply placed on the palm side of the arm, in reality, the blood pressure measuring apparatus 100 is wound around the arm.

The blood pressure measuring apparatus 100 includes the sensing device 110 and the calibration device 150. The sensing device 110 includes the pressure pulse wave sensor 111, a clocking unit 112, a pressing unit 113, a pulse wave measuring unit 114, a pump and valve 115, a pressure sensor 116, a communication unit 117, an operation unit 118, a display 119, an electric power source unit 120, a blood pressure calculator 121, a calibrator 122, a memory device 123, an ID (identification information) determination unit 124, and an ID memory 125. The calibration device 150 includes a communication unit 151, an electric power source unit 165, a blood pressure measuring unit 155, a pump and valve 156, a pressure sensor 157, a cuff 158, a display 162, an operation unit 163, a clocking unit 164, and an ID memory 166.

The blood pressure measuring apparatus 100 is annular and is wound around the wrist, etc., like a bracelet to measure a blood pressure based on biological information. The sensing device 110 is placed on the wrist closer to the palm than the calibration device 150, as shown in FIGS. 2 and 3. In other words, the sensing device 110 is placed farther from the elbow than the calibration device 150. In the present embodiment, the sensing device 110 is placed in such a manner that the pressure pulse wave sensor 111 is positioned above the radial artery, and consistent with this placement, the calibration device 150 is placed closer to The elbow than the sensing device 110. The sensing device 110 and the calibration device 150 may be worn on different arms. It is generally preferable to place the sensing device 110 and the calibration device 150 at the same height. It is further preferable to place the sensing device 110 and the calibration device 150 as the height of the heart.

The sensing device 110 has length L₁ in the extending direction of the arm, while the calibration device 150 has length L in the extending direction. The length L₁ is set smaller than length L2. The length L₁ of the pulse wave detector 110 in the extending direction of the arm is set to 40 mm or less, more desirably, 15 to 25 mm. The sensing device 110 has length W₁ in the direction perpendicular to the extending direction of the arm, while the calibration device 150 has length W₂ in the direction perpendicular to the extending direction. The length W₁ is set between 4 and 5 cm, while the length W₂ is set between 6 and 7 cm. Furthermore, the length W₁ and the length W₂ have a relationship of 0 (or 0.5) cm<W₂−W₁<2 cm. This relationship sets W₂ not to be too long, and makes it difficult to interfere with surroundings. By setting the size of the sensing device 110 within such a range, the calibration device 150 is placed closer to the palm so that the pulse wave can be easily detected and the measurement accuracy can be maintained. However, the calibration device 150 may be placed on the upper arm during measurement.

The pressure pulse wave sensor 111 detects the pressure pulse wave in a temporally continuous manner. For example, the pressure pulse wave sensor 111 detects a pressure pulse wave for each pulse. The pressure pulse wave sensor 111 is placed on the palm side, as shown in FIG. 2, and is usually placed parallel to the extending direction of the arm as shown in FIG. 3. With the pressure pulse wave sensor 111, time-series data of a blood pressure value (blood pressure waveform) that varies in conjunction with a heart rate can be obtained.

The clocking unit 112 outputs a time to the pressure pulse wave sensor 111. The clocking unit 112 allows the pressure pulse wave sensor 111 to pass data on the pressure pulse wave as well as the time to another component. For example, the memory device 123 records a time as well as data to be stored.

The pressing unit 113 is an air bag that presses the sensor portion of the pressure pulse wave sensor 111 against the wrist, thereby increasing the sensitivity of the sensor.

The pulse wave measuring unit 114 receives pressure pulse wave data from the pressure pulse wave sensor 111, together with the time, and passes the received data to the blood pressure calculator 121 and the memory device 123. The pulse wave measuring unit 114 controls the pump and valve 115 and the pressure sensor 116 to pressurize or depressurize the pressing unit 113, and adjusts the pressure pulse wave sensor 111 so as to press the radial artery at the wrist.

The communication unit 117 and the communication unit 151 communicate with each other by a communication system that enables short-distance data exchange. These communication units use, for example, a short-distance wireless communication system, such as Bluetooth (registered trademark), TransferJet (registered trademark), ZigBee (registered trademark), and IrDA (registered trademark).

The pump and valve 115 pressurizes or depressurizes the pressing unit 113, according to an instruction from the pulse ware measuring unit 114. The pressure sensor 116 monitors the pressure of the pressing unit 113, and notifies the pulse wave measuring unit 114 of the pressure value of the pressing unit 113.

The electric power source unit 120 supplies electric power to each component of the sensing device 110.

The ID memory 125 pre-stores identification information (also referred to as ID information) of the calibration device 150 to be paired with the sensing device 110. This identification information is used when pairing is established with the calibration device 150. The sensing device 110 receives data from the calibration device 150 stored in the ID memory 125. The “pairing” is also referred to as the calibration device 150 corresponding to the sensing device 110, or the sensing device 110 corresponding to the calibration device 150.

The blood pressure measuring unit 155 measures the blood pressure, which is biological information, with higher accuracy than the pressure pulse wave sensor 111. The blood pressure measuring unit 155, for example, measures the blood pressure intermittently, not continuously in terms of time, and transmits the measured value to the memory device 123 and the calibrator 122 via the corner communication units 151 and 117. The blood pressure measuring unit 155 measures the blood pressure using, for example, an oscillometric technique. Moreover, the blood pressure measuring unit 155 controls the pump and valve 156 and the pressure sensor 157 to pressurize or depressurize the cuff 158, thereby measuring the blood pressure. The blood pressure measuring unit 155 transmits, to the memory device 123 via the communication units 151 and 117, a systolic blood pressure together with a time when the systolic blood pressure is measured, and a diastolic blood pressure together with a time when the diastolic blood pressure is measured. The systolic blood pressure is also referred to as “SBP”, and the diastolic blood pressure is also referred to as “DBP”.

The memory device 123 sequentially acquires from the pulse wave measuring unit 114, pressure pulse wave data together with a detection time, and stores them. The memory device 123 also acquires from the blood pressure measuring unit 155 via the communication units 151 and 117, an SBP measurement time together with the SBP, as well as a DBP measurement time together with the DBP, acquired when the measuring unit operates. In addition, for type information and/or unique identification information of the calibration device which is a measuring instrument of the first biological information (measured by blood pressure measuring unit 155) for calibration used to calculate the measured biological information (continuous blood pressure), the memory device 123 keeps track of the information in association with the measured biological information. It is thereby possible to know, from the measured biological information, which blood pressure monitor (model, unique device number, etc.) has been used for the calibration.

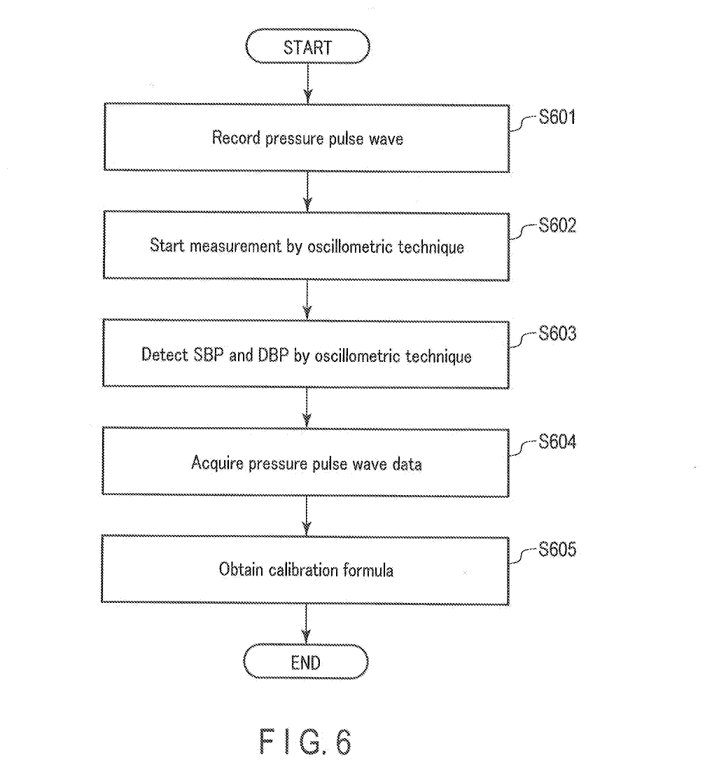

The calibrator 122 acquires, from the memory device 123, the SBP and DBP measured by the blood pressure measuring unit 155 together with the measurement time, and the data on the pressure pulse wave measured by the pulse wave measuring unit 114 of the sensing device 110 together with the measurement time. The calibrator 122 calibrates the pressure pulse wave from the pulse wave measuring unit 114, based on the blood pressure value from the blood pressure measuring unit 155. Of several calibration techniques that may be adopted by the calibrator 122, an example calibration technique will be described later in detail, with reference to FIG. 6.

The blood pressure calculator 121 receives the calibration method from the calibrator 122, calibrates the pressure pulse wave data from the pulse wave measuring unit 114, and causes the memory device 123 to store the blood pressure data obtained from the calibrated pressure pulse wave data, together with the measurement time.

The electric power source unit 165 supplies electric power to each component of the calibration device 150.

The display 162 displays various types of information, such as the result of the blood pressure measurement, to the user. For example, the display 162 receives data from the blood pressure measuring unit 155 and displays the contents of the data. For example, the display 162 displays the blood pressure value data with the measurement time.

The display 119 also displays various types of information, such as the result of the blood pressure measurement, to the user. For example, the display 119 receives data from the pulse wave measuring unit 114 and displays the contents of the data. For example, the display 119 displays the pressure pulse wave data together with the measurement time.

The operation unit 163 receives operation by the user. The operation Unit 168 includes, for example, an operation button for causing the blood pressure measuring unit 155 to start measurement, an operation button for performing calibration, and an operation button for starting or terminating communication.

The operation unit 118 also receives operation by the user. The operation unit 118 includes, for example, an operation button for causing the pulse wave measuring unit 114 to start measurement, and an operation button for starting or terminating communication.

The clocking unit 164 generates and supplies a time to a Component requiring it .

The ID memory 166 pre-stores identification information of the calibration device 150.

The ID determination unit 124 determines whether the ID contained in the data from the calibration device 150 is stored in the ID memory 125 of the sensing device 110, determines, the ID is one stored in the ID memory 125, that this data is data transmitted from the valid calibration device 150, and instructs the sensing device 110 to accept this blood pressure value data.

At the time of implementation, a program for executing each of the above-described operations is stored in, for example, a secondary storage device included in each of the pulse wave measuring unit 114, the calibrator 122, theblood pressure calculator 121, and the blood pressure measuring unit 155, and the central processing unit (CPU) executes a read operation of the stored program. The secondary storage device may be, for example, a hard disk, but any storage device can be used, such as a semiconductor memory, a magnetic storage device, an optical storage device, an optical magnetic disk, and a storage device to which phase change recording technology is applied.

In addition, programs for executing operations to be performed by the pulse wave measuring unit 114, the calibrator 122, the blood pressure calculator 121, and the blood pressure measuring unit 155 may be stored ire a server, etc., separate from the sensing device and the calibration device, and the stored programs may be executed therein. In this case, the pulse wave data measured by the sensing device and the blood pressure data measured by the calibration device as biological information may be transmitted to and calibrated by the server, thus allowing the server to obtain the blood pressure based on the pulse wave. Since the processing is performed by the server in such a case, the processing speed may increase. Moreover, because the device pay is of the pulse wave measuring unit 114, the calibrator 122, the blood pressure calculator 121, and the blood pressure measuring unit 155 are removed from the sensing device and the calibration device, the sizes thereof can be reduced, and the sensor can be easily placed at a position where accurate measurement can be performed. This reduces the burden on the user, leading to simple and accurate blood pressure measurement.

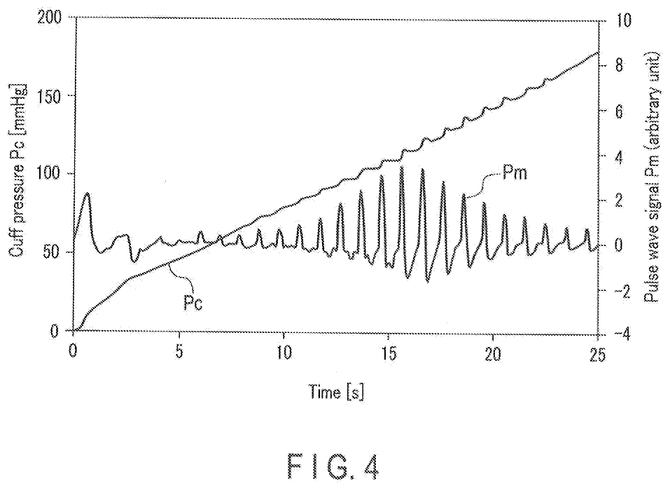

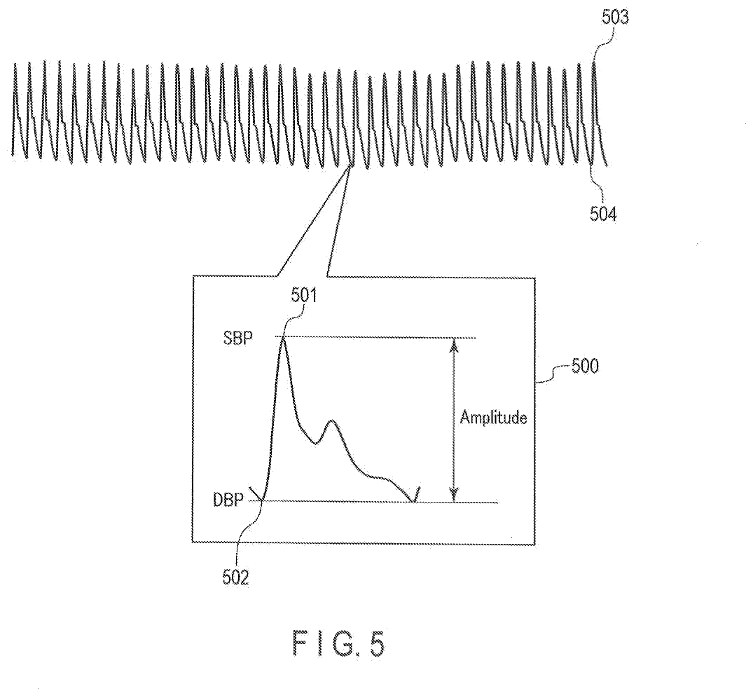

Next, operations performed by the pulse wave measuring unit 114 and the blood pressure measuring unit 155 prior to calibration by the calibrator 122 will be described with reference to FIGS. 4 and 5. FIG. 4 shows the time variation of the cuff pressure and the time variation of the magnitude of the pulse wave signal in the blood pressure measurement by the oscillometric technique. FIG. 4 shows the time variation of the cuff pressure and the time variation of the pulse wave signal, and illustrates that the cuff pressure increases with time while the magnitude of the pulse wave signal gradually increases to the maximum value with the increase of the cuff pressure and then decreases gradually. FIG. 5 shows time-series data of the pulse pressure at the time when the pulse pressure is measured for each pulse. Furthermore, FIG. 5 shows the waveform of one of the pressure pulse waves.

First, the operation at the time when the wrist blood pressure measuring unit 155 performs blood pressure measurement by the oscillometric technique will be briefly described with reference to FIG. 4. A blood pressure value may be calculated not only in a pressurization process but also in a pressure reduction process; however, only the pressurization process is described herein.

When a user instructs blood pressure measurement by oscillometric technique using the operation unit 163 provided in the calibration device 150, the blood pressure measuring unit 155 starts operation to initialize the processing memory area. Moreover, the blood pressure measuring unit 155 deactivates the pump of the pump and valve 156 to open the valve, thereby exhausting the air in the cuff 158. Subsequently, control is performed to set a current output value of the pressure sensor 157 as a value corresponding to the atmospheric pressure (adjusted to 0 mmHg).

Thereafter, the blood pressure measuring unit 155 functions as a pressure controller, and performs control to deliver air to the cuff 158 by closing the valve of the pump and valve 156 and then driving the pump. This expands the cuff 158, and gradually increases the cuff pressure (Pc in FIG. 4) to apply pressure. In this pressurization process, the wrist blood pressure measuring unit 155 monitors the cuff pressure Pc via the pressure sensor 157 in order to calculate a blood pressure value, and acquires, as a pulse wave signal Pm as shown in FIG. 4, a fluctuation component of the arterial volume generated in the radial artery in the wrist as a site to be measured.

Next, based on the pulse wave, signal Pm acquired at this time, the blood pressure measuring unit 155 attempts to calculate the blood pressure values (SBP and DBP) by applying a known algorithm by the oscillometric technique. If the blood pressure value cannot be calculated at this point because of insufficient data, as long as the cuff pressure Pc has not reached the upper limit pressure (predetermined to be, for example, 300 mmHg, for safety), the pressurization processing same as above is repeated.

After the blood pressure values are thus calculated, the blood pressure measuring unit 155 performs control to exhaust the air in the cuff 158, by deactivating the pump of the pump and valve 156 to open the valve. Lastly, the measurement result of the blood pressure value is passed to the calibrator.

Next, with reference to FIG. 5, a description will be given of the pulse wave measuring unit 114 that measures the pulse wave for each pulse. The pulse wave measuring unit 114 measures the pulse wave by, for example, tonometry method.

The pulse wave measuring unit 114 controls the pump and valve 115 and the pressure sensor 116 so that the pressure pulse wave sensor 111 obtains the optimum pressing force determined in advance to realize the optimum measurement, and increases the internal pressure of the pressing unit 113 to the optimum pressing force and holds it. Next, when a pressure pulse wave is detected by the pressure pulse wave sensor 111, the pulse wave measuring unit 114 acquires this pressure pulse wave.

The pressure pulse wave is detected for each pulse as a waveform as shown in FIG. 5, and each pressure pulse wave is detected continuously. In FIG. 5, the pressure pulse wave 500 is a pressure pulse wave of one pulse. The pressure value of 501 corresponds to SBP, and the pressure value of 502 corresponds to DBP. As shown in the pressure pulse wave time series of FIG. 5, normally, SBP 503 and DBP 504 fluctuate for each pressure pule wave.

Next, the operation of the calibrator 122 will be described with reference to FIG. 6.

The calibrator 122 calibrates the pressure pulse wave detected by the pulse wave measuring unit 114 using a blood pressure value measured by the blood pressure measuring unit 155. That is, the calibrator 122 determines blood pressure values of the maximum value 501 and the minimum value 502 of the pressure pulse wave detected by the pulse wave measuring unit 114.

### Calibration Method

The pulse wave measuring unit 114 starts recording the pressure pulse wave data together with the measurement time of the pressure pulse wave, and sequentially stores the pressure pulse wave data in the memory device 123 (step S601). Thereafter, for example, a user activates the blood pressure measuring unit 155 using the operation unit 163 to start measurement by the oscillometric technique (step S602). Based on the pulse wave signal Pm, the blood pressure measuring unit 155 records SBP data and DBP data, together with the times when the SBP and DBP are detected by the oscillometric technique, and stores these SBP data and DBP data in the memory device 123 (step S603).

The calibrator 122 acquires pressure pulse waves corresponding to the SBP data and the DBP data from the pressure pulse wave data (step S604). The calibrator 122 obtains a calibration formula based on the maximum value 501 of the pressure pulse wave corresponding to SBP and the minimum value 502 of the pressure pulse wave corresponding to DBP (step S605).

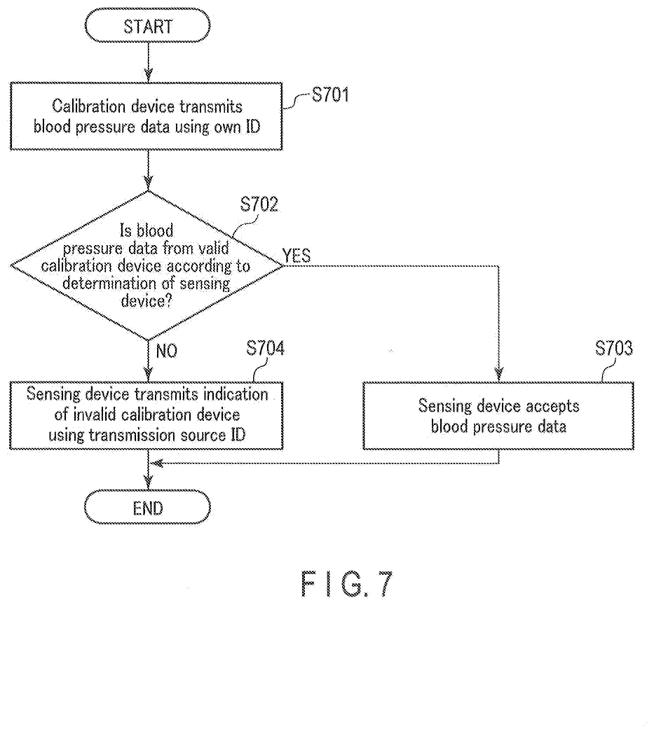

Next, with reference to FIG. 7, a description will be given of the operation in which when the sensing device 110 of the blood pressure measuring apparatus of the present embodiment receives blood pressure data which is data of the blood pressure varies from the calibration device 150 of the blood pressure measuring apparatus, the sensing device checks whether the blood pressure data is data from the intended calibration device 150 corresponding to the sensing device 110.

The sensing device 110 and the calibration device 150 of the present embodiment pre-store identification information of their own, and identification information of the apparatus to be paired with as a partner. The apparatus to be paired with is the sensing device 110 or the calibration device 150 worn by the same biological body and detecting biological information of the same biological body. For example, in the sensing device 110, the ID memory 125 pre-stores its own ID and the ID of the calibration device 150 of the partner, while in the calibration device 150, the ID memory 166 pre-stores its own ID and the ID of the calibration device 150 of the partner. Here, however, the blood pressure data from the valid calibration device 150 can be acquired by only storing the ID of the corresponding calibration device 150 in the ID memory 125.

First, the calibration device 150 transmits blood pressure data using its own ID (step S701). For example, the calibration device 150 adds its own ID to blood pressure data and transmits it. The sensing device 110 receives data from the calibration device 150, and extracts an ID contained in the data, and the ID determination unit 124 determines whether this ID is one pre-stored in the ID memory 125, and determines whether this blood pressure data comes from previously-Set calibration device 150 (step S702). If the ID contained in the data from the calibration device 150 is one stored in the ID memory 125, it is determined that the blood pressure data is data transmitted from the valid calibration device 150, and the sensing device 110 accents the blood pressure data (step S703). The sensing device 110 may return an acknowledgment, indicating that the blood pressure data has been accepted, to the calibration device 150.

On the other hand, if the ID contained in the data from the calibration device 150 is not one stored in the ID memory 125, it is determined that the blood pressure data is not data transmitted from the valid calibration device 150 (step S704). In this case, moreover, the sensing device 110 transmits, to this transmission source, an indication that it is an invalid calibration device using the transmission source ID transmitted together with the blood pressure data (step S704). The transmission source calibration device receives the indication of the invalid calibration device, knowing that the blood pressure data transmitted by itself is not subjected to the calibration.

According to the first embodiment described above, since the sensing device 110 and the calibration device 150 are separated, the necessity to align the calibration device 150 is eliminated, and the pressure pulse wave sensor 111 of the sensing device 110 can be placed at the optimum position. The pulse wave is calibrated based on the first blood pressure value measured by the calibration device 150, and the second blood pressure value is calculated from the pulse wave. This makes it possible to calculate biological information with high accuracy based on the pulse wave, allowing the user to easily obtain highly accurate biological information. Moreover, since the calibration device 150 is independently provided, the calibration device can be mounted at a position appropriate for calibration with ease, regardless of the placement of the sensing device 110. In addition, each of the apparatuses pre-stores its own ID and the partner's ID, making it possible for apparatus performing calibration to acquire blood pressure data from a valid paired partner apparatus and to avoid erroneous use of data from a non-paired partner.

### Second Embodiment

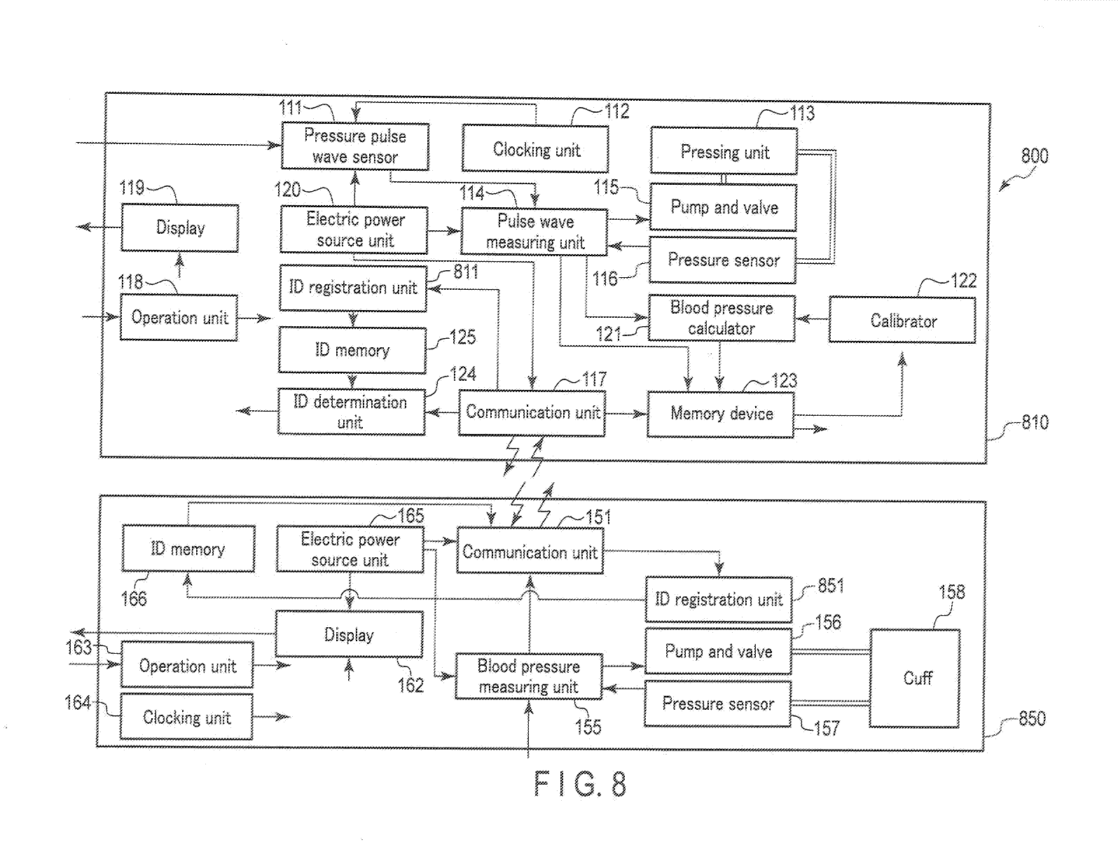

The blood pressure measuring apparatus 800 according to the second embodiment will be described with reference to FIGS. 8, 2 and 3. FIG. 8 is a functional block diagram of the blood pressure measuring apparatus 800, and illustrates details of a sensing device 810 and a calibration device 850. FIG. 2 shows an example in which the blood pressure measuring apparatus 100 is worn on the wrist, and is a schematic perspective view seen from above the palm. The same applies to the blood pressure measuring apparatus 800. The pressure pulse wave sensor 111 is placed on the wrist side of the sensing device 110. FIG. 3 is a schematic perspective view conceptually illustrating the blood pressure measuring apparatus 100 when worn, as seen from the lateral side of the palm (i.e., the direction in which the fingers are aligned when the hand is open). The same applies to the blood pressure measuring apparatus 800. FIG. 3 shows an example in which the pressure pulse wave sensor 111 is placed orthogonal to the radial artery. Although in FIG. 3, the blood pressure measuring apparatus 100 appears to be simply placed on the palm side of the arm, in reality, the blood pressure measuring apparatus 100 is wound around the arm. FIGS. 2 and 3 apply to the present embodiment, similarly to the first embodiment.

The blood pressure measuring apparatus 800 according to the present embodiment is different from the blood pressure measuring apparatus 100 according to the first embodiment as regards a sensing device 810 and a calibration device 850.

The sensing device 810 of the present embodiment corresponds to the sensing device 110 of the first embodiment, but includes an ID registration unit 811. The ID registration unit 811 registers an ID of the calibration device 850 to be paired with.

The calibration device 850 of the present embodiment corresponds to the calibration device 150 of the first embodiment, but includes an ID registration unit 851. The ID registration unit 851 registers an ID of the sensing device 810 to be paired with.

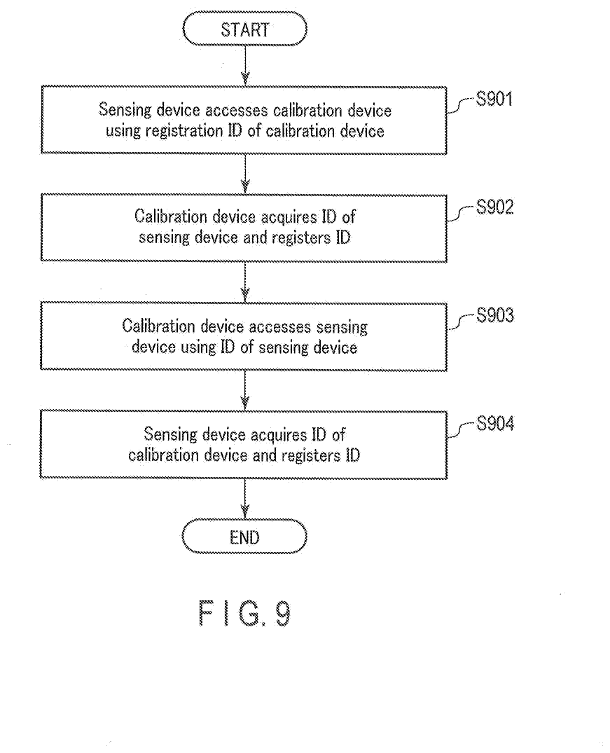

Next, with reference to FIG. 9, a description will be given of an operation in which each of the sensing device 810 and the calibration device 850 registers the ID of the partner apparatus.

The sensing device 810 accesses the calibration device 850 using the registration ID of the calibration device 850 (step S901). In this step, the sensing device 810 is connected to the calibration device 850 to provide the calibration device 850 with identification information of the sensing device 810. Then, the calibration device 850 acquires an ID of the sensing device 810, and the ID registration unit 851 registers this ID in the ID memory 166 (step S902).

The calibration device 850 accesses the sensing device 810 using the ID of the sensing device 810 (step S903). In this step, the calibration device 850 is connected to the sensing device 810 is to provide the sensing device 810 with identification information of the calibration device 850. Then, the sensing device 810 acquires an ID of the calibration device 850, and the ID registration unit 811 registers this ID in the ID memory 125 (step S904).

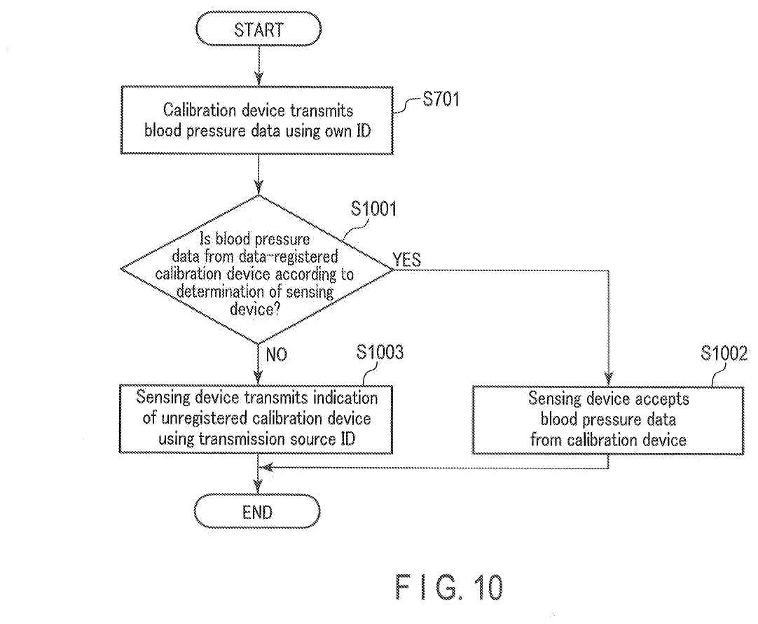

Next, with reference to FIG. 10, a description will be given of the operation in which the calibration device 850 transmits blood pressure data., and the sensing device 810 receives blood pressure data. FIG. 10 shows the operation in which when the blood pressure data is transmitted to the sensing device 810 of the blood pressure measuring apparatus, the sensing device checks whether the blood pressure data is data from the intended calibration device 850 corresponding to the sensing device 810.

First, as described with reference to FIG. 7, the calibration device 850 transmits blood pressure data using its own ID (step S701). The sensing device 810 receives data from the calibration device 850, and extracts an ID transmitted together with the data, and the ID determination unit 124 determines whether this ID is one registered in the ID memory 125, and determines whether this blood pressure data is from the registered calibration device 850 (step S1001).

If the ID transmitted together with the data from the calibration device 850 is one registered n the ID memory 125, it is determined that the blood pressure data is data transmitted from the valid calibration device 850, and the sensing device 810 accepts this blood pressure data (step S1002). The sensing device 810 may return an acknowledgment, indicating that the transmitted blood pressure data has been accepted, to the calibration device 850.

On the other hand, if the ID transmitted together with the data from the calibration device 850 is not one stored in the ID memory 125, it is determined that this blood pressure data is not data transmitted from the valid calibration device 850 (step S1003). Further, in this the sensing device 810 transmits to this transmission source an indication that the apparatus is an unregistered calibration device, using the transmission source ID transmitted together with the blood pressure data (step S704). The transmission source calibration device receives the indication of the unregistered calibration device, knowing that the blood pressure data transmitted by itself is not subjected to the calibration.

In addition to the advantageous effects of the first embodiment, according to the second embodiment described above, it is possible to acquire the pulse wave data or the blood pressure data from the valid paired partner apparatus and to avoid erroneous use of data from a non-paired partner, because the sensing device 810 and the calibration device 850 register the ID of the partner to be paired with.

### Third Embodiment

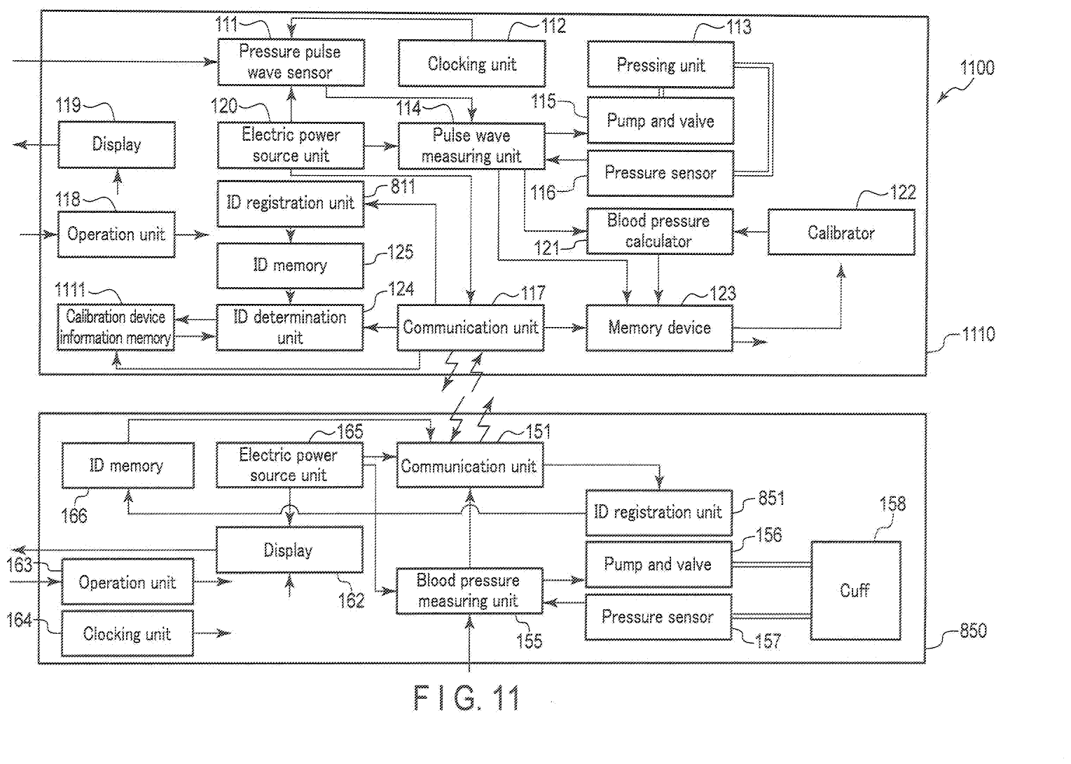

The blood pressure measuring apparatus 1100 according to the third embodiment will be described with reference to FIGS. 11, 2, and 3. FIG. 11 is a functional block diagram of the blood pressure measuring apparatus 1100, and illustrates details of a sensing device 1110 and a calibration device 850. FIG. 2 shows an example in which the blood pressure measuring apparatus 100 is worn on the wrist, and is a schematic perspective view seen from above the palm. The same applies to the blood pressure measuring apparatus 1100. The pressure pulse wave sensor 111 is placed on the wrist side of the sensing device 1110. FIG. 3 is a schematic perspective view conceptually illustrating the blood pressure measuring apparatus 100 when worn, as seen from the lateral side of the palm (i.e., the direction in which the fingers are aligned when the hand is open). The same applies to the blood pressure measuring apparatus 1100. FIG. 3 shows an example in which the pressure pulse wave sensor 111 is placed orthogonal to the radial artery. Although in FIG. 3, the blood pressure measuring apparatus 100 appears to be simply placed on the palm side of the arm, in reality, the blood pressure measuring apparatus 100 is wound around the arm. FIGS. 2 and 3 apply to the present embodiment, similarly to the first embodiment.

The blood pressure measuring apparatus 1100 of the present embodiment is different from the blood pressure measuring apparatus 800 according to the second embodiment only as regards the sensing device 1110.

The sensing device 1110 of the present embodiment corresponds to the sensing device 810 of the second embodiment, but includes a calibration device information memory 1111. The calibration device information memory 1111 pre-stores unique information such as an ID of a calibration device that can be used as a calibration device. The ID determination unit 124 receives an ID acquired from the ID memory 125, and determines whether this ID is pre-stored in the calibration device information memory 1111. If the ID is one pre-stored in the calibration device information memory 1111, the ID determination unit 124 determines that the calibration device of the current partner is a valid apparatus. On the other hand, if the received ID is not one pre-stored in the calibration device information memory 1111, it is determined that the calibration device of the current partner is not a valid apparatus and that calibration should not be performed by this calibration device. Moreover, if the received ID is not one pre-stored in the calibration device information memory 1111, an indication may be transmitted to the calibration device that the calibration device is not one to be paired with the sensing device, using the acquired ID of the calibration device.

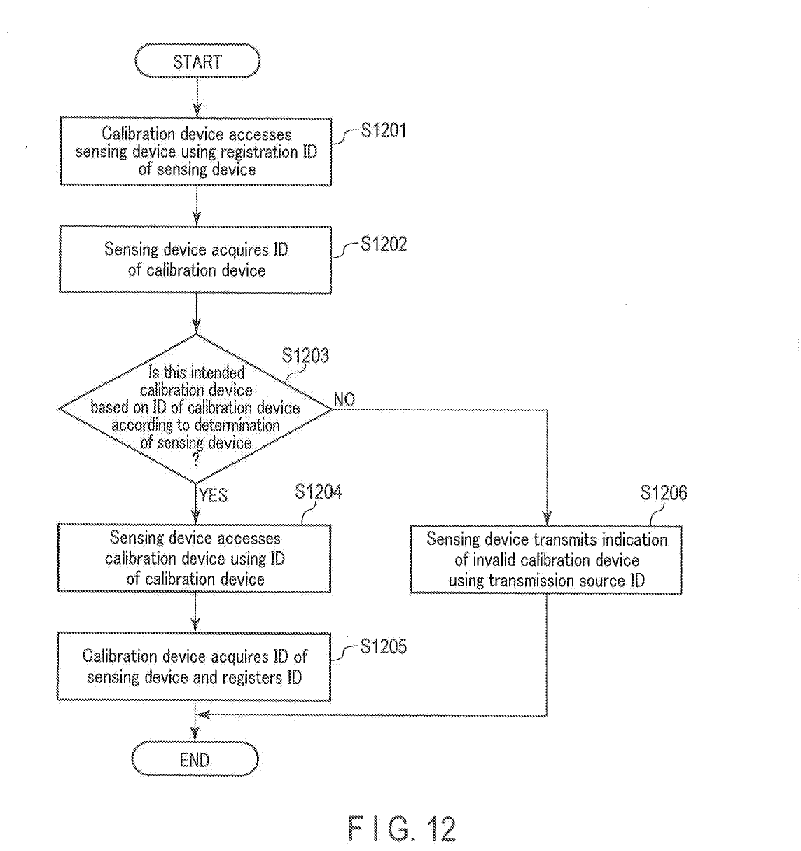

Next, with reference FIG. 12, a description will be given of the operation in which the sensing device 1110 selects the calibration device 850 to be paired with the sensing device 1110.

The calibration device 850 accesses the sensing device 1110 using the registration ID of the sensing device 1110 (step S1201). In this step, the calibration device 850 is connected to the sensing device 1110 to provide the sensing device 1110 with identification information of the calibration device 850. Then, the sensing device 1110 acquires the ID of the calibration device 850, and the ID registration unit 811 passes this ID to the ID memory 125 (step S1202). Next, the ID determination unit 124 determines whether the ID of the calibration device 850 matches any one of the IDs pre-stored in the calibration device information memory 1111, and if it is determined that there is a matching ID, the processing advances to step S1204, and if it is determined that there is no matching ID, the processing advances to step S1206 (step S1203).

The sensing device 1110 accesses the calibration device 850 using the ID of the calibration device 850 (step S1204). In this step, the sensing device 1110 is connected to the calibration device 850 to provide the calibration device 850 with identification information of the sensing device 1110. Then, the calibration device 850 acquires an ID of the sensing device 1110, and the ID registration unit 851 registers the ID in the ID memory 166 (step S1205). On the other hand, in step S1206, the sensing device 1110 transmits an indication of in lid calibration device, using the ID of the calibration device 850. Thus, the sensing device 1110 can exchange identification information with the calibration device 850 to be paired with, allowing data exchange between appropriate valid apparatuses.

In addition to the advantageous effects of the first embodiment, according to the third embodiment described above, because the sensing device 1110 pre-stores a list of calibration devices to be calibrated and determines whether the ID of the calibration device is in this list, it is possible to determine a calibration device to be paired with and to avoid data exchange with a wrong apparatus, allowing accurate calibration. As a result, according to the present embodiment, it is possible to measure blood pressure with high accuracy in a temporally continuous manner.

### Fourth Embodiment

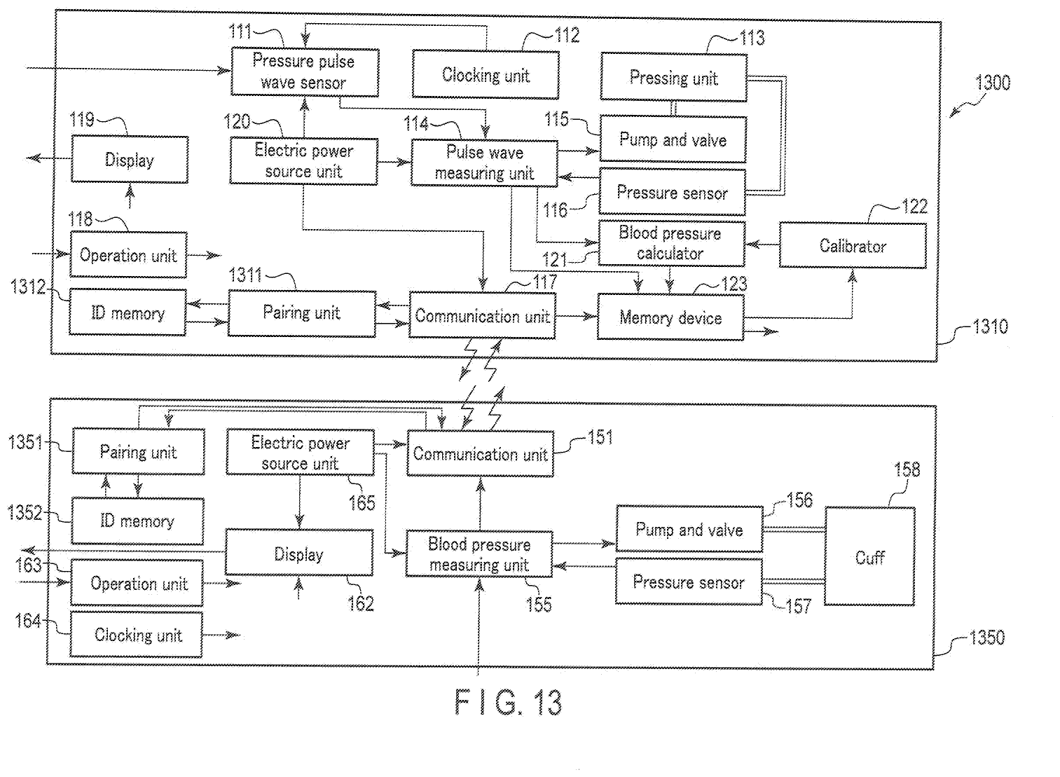

The blood pressure measuring apparatus 1300 according to the fourth embodiment will be described with reference to FIGS. 13, 2, and 3. FIG. 13 is a functional block diagram of the blood pressure measuring apparatus 1300, axed illustrates details of a sensing device 1310 and a calibration device 1350. FIG. 2 shows an example in which the blood pressure measuring apparatus 100 is worn on the wrist, and is a schematic perspective view seen from above the palm. The same applies to the blood pressure measuring apparatus 1300. The pressure pulse wave sensor 111 is placed on the wrist side of the sensing device 110. FIG. 3 is a schematic perspective view conceptually illustrating the blood pressure measuring apparatus 100 when worn, as seen from the lateral side of the palm (i.e., the direction in which the fingers are aligned when the hand is open). The same applies to the blood pressure measuring apparatus 1300. FIG. 3 shows an example in which the pressure pulse wave sensor 111 is placed orthogonal to the radial artery. Although in FIG. 3, the blood pressure measuring apparatus 100 appears to be simply placed on the palm side of the arm, in reality, the blood pressure measuring apparatus 100 is wound around the arm. FIGS. 2 and 3 apply to the present embodiment, similarly to the first embodiment.

The blood pressure measuring apparatus 1300 according to the present embodiment is different from the blood pressure measuring apparatus 100 according to the first embodiment as regards a sensing device 1310 and a calibration device 1350.

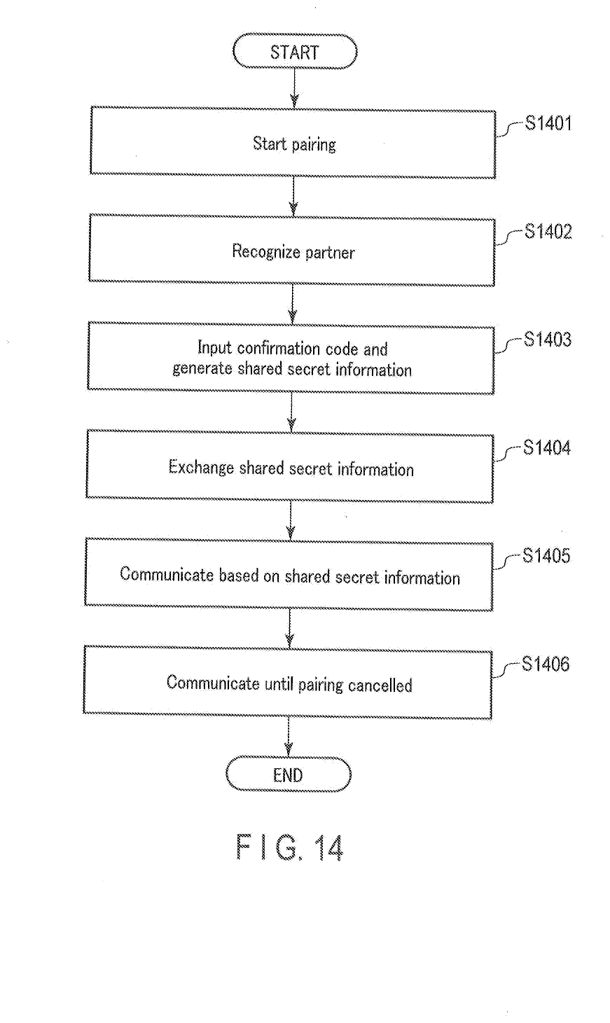

The sensing device 1310 of the present embodiment corresponds to the sensing device 110 of the first embodiment, but includes a pairing unit 1311 and an ID memory 1312 instead of the ID memory 125. The pairing unit 1311 executes an operation for pairing with the calibration device 1350. Specifically, the pairing unit 1311 performs the operation shown in FIG. 14. The ID memory 1312 stores, for example, identification information of the sensing device 1310, a confirmation code by pairing operation, and shared secret information.

The calibration device 1350 of the present embodiment corresponds to the calibration device 150 of the first embodiment, but includes a pairing unit 1351 and an ID memory 1352 instead of the ID memory 166. The pairing unit 1351 executes an operation for pairing with the sensing device 1310 and performs the same operation as the pairing unit 1311. The ID memory 1312 stores, for example, identification information of the calibration device 1350, a verification code by pairing operation, and shared secret information.

Next, an operation of pairing the sensing device 1310 and the calibration device 1350 will be described with reference to FIG. 14. The operation shown in FIG. 14 is performed by the sensing device 1310 and the calibration device 1350 in a similar manner, thus, the “apparatus” referred to in the following description with reference to FIG. 14 indicates both the sensing device 1310 and the calibration device 1350.

Both apparatuses start pairing (S1401). For example, both apparatuses emit short-distance radio waves including their own identification information. When both apparatuses receive the radio waves, they recognize their partner apparatuses (step S1402). The apparatus checks whether the recognized partner is an intended partner, and if so, accepts a confirmation code, and generates shared secret information based on the code (step S1403). Whether it is the intended partner may be determined, for example, by registering in the ID memories 1312 and 1352 an ID of a device exceeding criteria as a pairing partner, and determining whether the memories have the ID (identification information). In addition, it may determined to pair with a partner by including information indicating specifications (specs) or performance of the apparatus in identification information and notifying such to the partner apparatus and if it is indicated that the performance of the apparatus exceeds certain criteria based on the identification information.

Then, the shared secret information is exchanged between tie two apparatuses (step S1404). Thereafter, data is encrypted based on the shared secret information generated by one of the apparatuses and transmitted to the other apparatus, and data received from the partner apparatus is decrypted based on the shared secret information received from the partner apparatus (step S1405). Such communication operation is continued until pairing is cancelled (step S1406).

In addition to the advantageous effects of the first embodiment, according to the fourth embodiment described above, data can be freely exchanged with an intended partner by short-distance communication pairing. Therefore, even when the sensing device 1310 or the Calibration device 1350 cannot be used due to a failure or the like, it is possible to continue detection or measurement of biological information by pairing with other apparatus satisfying criteria such as performance.

### Fifth Embodiment

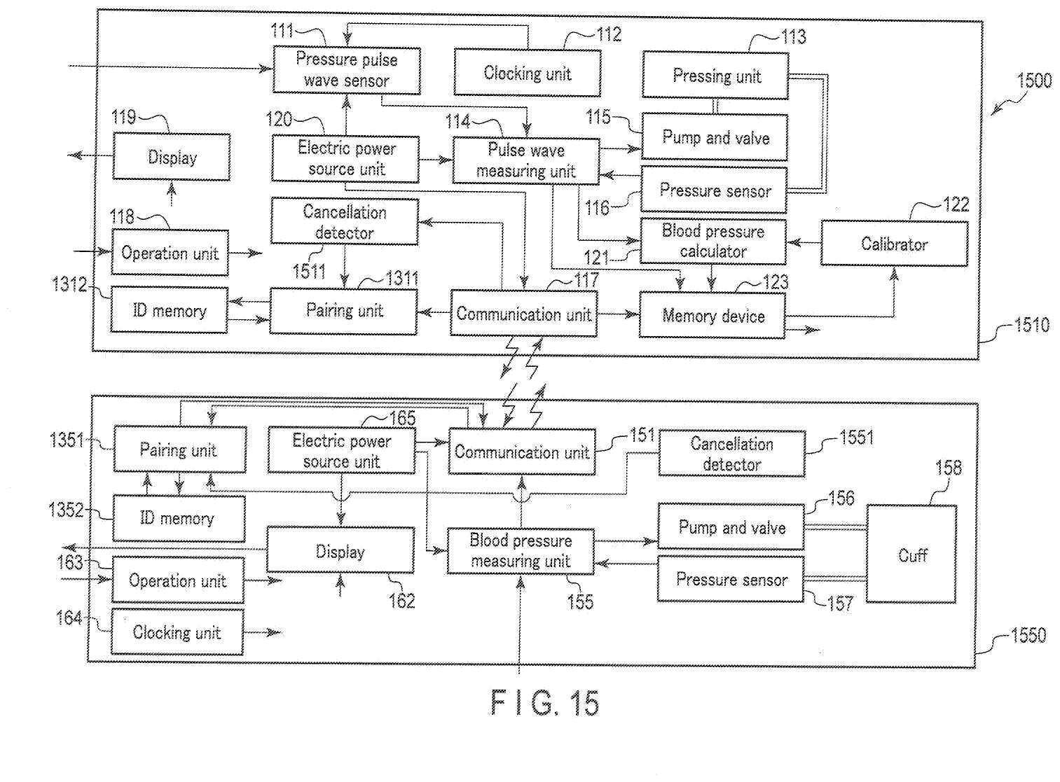

The blood pressure measuring apparatus 1500 according to the fifth embodiment will be described with reference to FIGS. 15, 2, and 3. FIG. 15 is a functional block diagram of the blood pressure measuring apparatus 1500, and illustrates details of a sensing device 1510 and a calibration device 1550. FIG. 2 shows an example in which the blood pressure measuring apparatus 100 is worn on the wrist, and is a schematic perspective view seen from above the palm. The same applies to the blood pressure measuring apparatus 1500. The pressure pulse wave sensor 111 is placed on the wrist side of the sensing device 110. FIG. 3 is a schematic perspective view conceptually illustrating the blood pressure measuring apparatus 100 when worn, as seen from the lateral side of the palm (i.e., the direction in which the fingers are aligned when the hand is open). The same applies to the blood pressure measuring apparatus 1500. FIG. 3 shows an example in which the pressure pulse wave sensor 111 is placed orthogonal to the radial artery. Although in FIG. 3, the blood pressure measuring apparatus 100 appears to be simply placed on the palm side of the arm, in reality, the blood pressure measuring apparatus 100 is wound around the arm. FIGS. 2 and 3 apply to the present embodiment, similarly to the first embodiment.

The blood pressure measuring apparatus 1500 according to the present embodiment is different from the blood pressure measuring apparatus 1300 according to the fourth embodiment as regards a sensing device 1510 and a calibration device 1550.

The sensing device 1510 of the present embodiment corresponds to the sensing device 1310 of the fourth embodiment, but includes a cancellation detector 1511. The cancellation detector 1511 monitors whether the pairing with the calibration device 1550 has been cancelled, and if the pairing has been cancelled, instructs the pairing unit 1311 to resume pairing.

The calibration device 1550 of the present embodiment corresponds to the calibration device 1350 of the fourth embodiment, but includes a cancellation detector 1551. The cancellation detector 1551 monitors whether the pairing with the sensing device 1510 has been cancelled, and if the pairing has been cancelled, instructs the pairing unit 1351 to resume pairing.

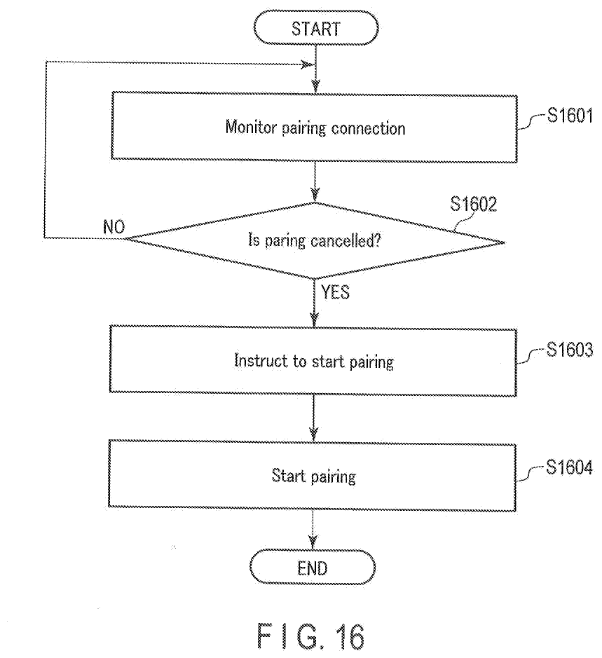

Next, with reference to FIG. 16, a description will be given of an operation of determining whether the pairing between the sensing device 1510 and the calibration device 1550 has been cancelled.

The cancellation detectors 1511 and 1551 of the respective sensing devices 1510 and calibration device 1550 monitor whether the connection by the pairing between the sensing device 1510 and the calibration device 1550 is continued, based on the information received from the communication units 117 and 151 (step S1601). Next, the cancellation detectors 1511 and 1551 determine whether the pairing has been cancelled, and if it is determined that the pairing has been cancelled, the processing proceeds to step S1603, and if it is determined that the pairing has not been cancelled, the processing returns to step S1601 to continue monitoring (step S1602). In step S1603, the cancellation detectors 1511 and 1551 instruct the respective pairing units 1311 and 1351 to start pairing (step S1603). The pairing units 1311 and 1351 start pairing according to the flowchart shown in FIG. 14 (step S1604).

In step S1603, normally, pairing with the partner connected immediately before is tried again. Identification information and the like are stared in the ID memories 1312 and 1352 so that the partner apparatus connected immediately before can be specified. For example, the ID memories 1312 and 1352 record identification information of the pairing partner, a connection start time, and a connection end time. In addition, if the pairing attempt in step S1604 fails more than a certain number of times, pairing with other calibration device may be tried with reference to the identification information of the calibration device in the ID memory 1312. In this case, for example, a pairing request is transmitted to the calibration device to have the pairing started.

In addition to the advantageous effects of the firs embodiment, according to the fifth embodiment described above, even if short- distance communication pairing is cancelled, pairing can be resumed automatically. Therefore, accurate detection and measurement of biological information can be performed in a temporally continuous manner with little interruption.

In the embodiments described above, the pressure pulse wave sensor 111 detects, for example, a. pressure pulse wave of the radial artery extending through a site to be measured (for example, the left wrist) (the tonometry method). However, the present invention is not limited to this. The pressure pulse wave sensor 111 may configured to detect the pulse wave of the radial artery passing through a measurement site (e.g., the left wrist) as a change in impedance (impedance method). The pressure pulse wave sensor 111 may include a light-emitting element that emits light toward an artery passing through the corresponding portion of the measurement site, and a light-receiving element that receives reflected light (or transmitted light) of the emitted light, and may be configured to detect arterial pulse waves as changes in volume (photoelectric method). Moreover, the pressure pulse wave sensor 111 may include a piezoelectric sensor in contact with the measurement site, and may be configured to detect a strain caused by the pressure of the artery passing through the corresponding portion of the measurement site as a change in electric resistance (piezoelectric method). Furthermore, the pressure pulse wave sensor 111 may include a transmit element that transmits radio waves toward an artery passing through the corresponding portion of the measurement site, and a receive element that receives reflection waves of the transmitted radio waves, and may be configured to detect a change in distance between the artery and the sensor caused by the pulse wave of the artery as a phase shift between the transmission waves and the reflection waves (radio wave irradiation method). Any method can be employed other than the above methods as long as the method enables observation of a physical quantity used to calculate the blood pressure.

In the above-described embodiment, the blood pressure measuring apparatuses 100, 800, 1100, 1300, and 1500 are assumed to be worn on the left wrist, which is the measurement site; however, the configuration is not limited thereto, and they may be worn on, for example, the right wrist. A site to be measured may be the upper limb such as the upper arm other than the wrist, or the lower limb such as the ankle or thigh as long as the site has an artery extending therethrough.

The apparatus of the present invention can also be realized by a computer and a program, and the program can be recorded on a recording medium or provided through a network.

Furthermore, each of the above-described devices and their device portions can be implemented either as a hardware configuration or as a combined configuration of hardware resources and software. The software of the combined configuration may be a program pre-installed in a computer from a network or a computer-readable storage medium to be executed by the processor of the computer for implementation of the functions of the respective apparatuses.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

A part or all of the above-mentioned embodiments may also be described as Ln the following additional notes, without limitation thereto.

### Additional Note 1

A biological information measuring apparatus comprising a sensing device including a first hardware processor and a calibration device including a second hardware processor and a memory,
the second hardware processor being configured to:
measure first biological information intermittently; and
transmit, to the sensing device, data including the first biological information and calibration identification information which is identification information of the calibration device,

the first hardware processor being configured to:
receive the, data and the calibration identification information;
determine whether the calibration identification information is information of a calibration device corresponding to the sensing device;
detect a pulse wave in a temporally continuous manner; and
calibrate, when the calibration identification information is information of the corresponding calibration device, the pulse wave based on the first biological information, and calculate second biological information based on the calibrated pulse wave, and
the memory comprising:
a memory device that stores the second biological information.

### Additional Note 2

A method of measuring biological information, comprising:

measuring first biological information intermittently using at least one hardware processor;

transmitting, to the sensing device, data including the first biological information and calibration identification information which is identification information of the calibration device using the at least one hardware processor;

receiving the data and the calibration identification information using the at least one hardware processor;

determining whether the calibration identification. information is information of a calibration device corresponding to the sensing device using the at least one hardware processor; and

calibrating, when the calibration identification information is information of the corresponding calibration device, the pulse wave based on the first biological information, and calculating second biological information based on the calibrated pulse wave, using the at least one hardware processor.

1. A biological information measuring apparatus. comprising a sensing device and a calibration device,
the calibration device Comprising:
a measuring unit configured to measure first biological information intermittently; and
a transmitter configured to transmit, to the Sensing device, data including the first, biological information, and calibration identification information which is identification information of the calibration device, and
the sensing device comprising:
a receiver configured to receive the data and the calibration identification information;
a determination unit configured to determine whether the calibration identification information is information of a calibration device corresponding to the sensing device;
a detector configured to detect a pulse wave in a temporally continuous manner; and
a calculator configured to, when the calibration identification information is information of a corresponding calibration device, calibrate the Pulse wave based on the first biological information, and calculate second biological information based on the calibrated pulse wave,

2. The apparatus according to claim 1, wherein the sensing device further comprises a sensor memory device configured to pre-store identification information of the corresponding calibration device, and
when the calibration identification information is included in the identification information stored in the sensor memory device, the determination unit determines that the calibration identification information is information of the corresponding calibration device.

3. The apparatus according to claim 1, wherein the sensing device further comprises a sensor registration unit configured to register identification information of the corresponding calibration device, and
when the calibration identification information is included in the identification information registered in the sensor registration unit, the determination unit determines that the calibration identification information is information of the corresponding calibration device.

4. The apparatus according to claim 1, further comprising a transmitter that, when the calibration identification information is determined to be not information of the calibration device corresponding to the sensing device, transmits an indication of invalid apparatus to the calibration device.

5. The biological information measuring apparatus according to claim 1, wherein
the sensing device further comprises:
a sensor memory that Stores identification information of the calibration device corresponding to the sensing device; and
a sensor pairing unit that emits a first radio wave, receives a second radio wave, and pairs with a calibration device emitting the second radio wave if identification information included in the second radio wave matches information in the sensor memory, and
the calibration device further comprises:
a calibration memory that stores identification information of a sensing device corresponding to the calibration device; and
a calibration pairing unit that emits the second radio wave, receives the first radio wave, and pairs with a sensing device emitting the first radio wave if identification information included in the first radio wave matches information stored in the calibration memory.

6. The biological information measuring apparatus according to claim 5, wherein the sensing device further comprises a sensor cancellation detector that determines whether pairing has been cancelled, and instructs the sensor pairing unit to start pairing when it is determined that pairing has been cancelled, and
the calibration device further comprises a calibration cancellation detector that determines whether pairing has been cancelled, and instructs the calibration pairing unit to start pairing when it is determined that pairing has been cancelled.

7. The biological information measuring apparatus according to claim 1, wherein the measuring unit measures the first biological information with higher accuracy than that of second biological information obtained from the detector.

8. The biological information measuring apparatus according to claim 1, wherein the detector detects the pulse wave for each pulse, and the first biological information and the second biological information indicate blood pressures.

9. A method of measuring biological information by a biological information measuring apparatus comprising a sensing device and a calibration device, the method comprising:
in the calibration device:
measuring first biological information intermittently; and
transmitting, to the sensing device, data including the first biological information, and calibration identification information which is identification information of the calibration device; and
in the sensing device:
receiving the data and the calibration identification information;
determining whether the calibration identification information is information of a calibration device corresponding to the sensing device;
detecting a pulse wave in a temporally continuous manner;
calibrating, when the calibration identification information is information of a corresponding calibration device, the pulse wave based on the first biological information; and
calculating second biological information based on the calibrated pulse wave.

10. A non-transitory computer readable medium storing a computer program which is executed by a computer to provide the steps of:
measuring first biological information intermittently; and
transmitting, to a sensing device, data including the first biological information, and calibration identification information which is identification information of a calibration device; and
receiving the data and the calibration identification information;
determining whether the calibration identification information is information of a calibration device corresponding to the sensing device;
detecting a pulse wave in a temporally continuous manner;
calibrating, when the calibration identification information is information of a corresponding calibration device, the pulse wave based on the first biological information; and
calculating second biological information based on the calibrated pulse wave.
